(19) United States
(12) Patent Application Publication
(10) Pub. No.: US 2017/0065311 A1
(43) Pub. Date: Mar. 9, 2017

(54) ANTERIOR CERVICAL PLATE

(71) Applicant: GS Medical Inc., King of Prussia, PA (US)

(72) Inventors: Milan George, Collegeville, PA (US); Seunghyun Yoon, Lansdale, PA (US)

(73) Assignee: GS Medical Inc., King of Prussia, PA (US)

(21) Appl. No.: 15/257,835

(22) Filed: Sep. 6, 2016

Related U.S. Application Data
(60) Provisional application No. 62/214,359, filed on Sep. 4, 2015.

(51) Int. Cl. A61B 17/80

(52) U.S. Cl. CPC A61B 17/8033; A61B 17/80; A61B 17/8061

(57) ABSTRACT

An anterior cervical plate assembly is characterized in that it comprises a base plate and fastener with a two-part locking mechanism. The locking mechanism includes a blocking plate that translates along a recess in the base plate to partially overlap a fastener, preventing back-out. The locking mechanism also includes a locking piece that extends through the base plate and has a rotational cam surface that causes the translation of the blocking plate.

### CROSS-REFERENCE TO RELATED APPLICATIONS

This application claims the benefit of U.S. Provisional Application No. 62/214,359, filed Sep. 4, 2015, the entire disclosure of which is incorporated by reference herein in its entirety.

### BACKGROUND OF THE INVENTION

The present invention relates generally to an anterior cervical plate assembly for attachment to one or more cervical vertebrae for the purpose of immobilizing, stabilizing and/or aligning those vertebrae. In particular, the present invention relates to an anterior cervical plate assembly with a locking mechanism that prevents bone screws from backing out of the plate.

Conventional anterior cervical plates include a base plate with a plurality of holes for receiving screws and screws for fastening to vertebrae. Such typical base plates extend across two or more vertebrae for the purpose of immobilizing, stabilizing and/or aligning those vertebrae. This is necessary for strength and rigidity after a fusion procedure, to secure vertebrae together after removal or replacement of intervening vertebrae or intervertebral discs, or to correct instability due to injury or disease. One failure mode of such conventional anterior cervical plates is unintentional back-out of bone screws over time, resulting in failure of the implant, further injury or at least incomplete transfer of loads between the base plate and the vertebrae.

### BRIEF SUMMARY OF THE INVENTION

In accordance with an exemplary embodiment, the present invention provides an anterior cervical plate assembly including a base plate, a blocking plate and a locking piece. The base plate has a recess. The blocking plate is positioned within the recess and has an aperture and a protrusion extending from a surface of the blocking plate forming the aperture. The locking piece is positioned within the aperture and has a cam for engaging the protrusion.

In accordance with another exemplary embodiment, the present invention provides an anterior cervical plate assembly having a base plate, a blocking plate and a locking piece. The base plate includes a pair of openings and a recess between the pair of openings. The blocking plate is positioned within the recess and has a through hole defining a cam follower. The pair of protrusions is positioned substantially diametrically opposed from each other. The locking piece is positioned within the aperture of the blocking plate and has a top portion, a reduced diameter portion adjacent the top portion, and a stem portion extending from the reduced diameter portion. The reduced diameter portion includes a cam.

In accordance with yet another exemplary embodiment, the present invention provides an anterior cervical plate assembly including a base plate, a blocking plate, a locking piece, and a locking ring. The base plate has a fastening aperture, first and second apertures for receiving bone screws, a top surface, a bottom surface, and an undercut. The fastening aperture includes a top recess, a bottom recess and an inner diameter. The undercut extends below the top surface and between the first and second apertures. The blocking plate has a blocking plate aperture, first and second protrusions extending into the blocking plate aperture, a relief cutout and a pair of outer corners. The blocking plate is positioned in the top recess and is configured to adopt an unlocked position wherein the first and second apertures are unobstructed. The blocking plate is configured to adopt a locked position obstructing the first and second apertures. The relief cutout is configured to slide under the undercut in the locked position. The pair of outer corners are configured to, in the locked position, obstruct one of the first and second base plate apertures. The locking piece has a top portion positioned upon the blocking plate, a cam-shaped portion and a lower portion having a reduced waist. The cam-shaped portion is configured for engaging the protrusions and the cam-shaped portion has a flat end. The locking ring is positioned within the bottom recess and is sized to encircle the reduced waist of the locking piece so as to prevent removal of the locking piece from the fastening aperture.

### BRIEF DESCRIPTION OF THE SEVERAL VIEWS OF THE DRAWINGS

The foregoing summary, as well as the following detailed description of the preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a perspective view of an anterior cervical plate assembly in accordance with an exemplary embodiment of the present invention with fasteners omitted for purposes of illustration;

FIG. 2A is a top perspective view of a base plate of the anterior cervical plate assembly of FIG. 1;

FIG. 2B is a bottom perspective view of the base plate of FIG. 2A;

FIG. 3A is a partial top plan view of an exemplary anterior cervical plate assembly in accordance with the present invention with an alternative embodiment of a blocking plate in a locked position;

FIG. 3B is a partial top plan view of an exemplary anterior cervical plate assembly in accordance with the present invention with an alternative embodiment of a blocking plate in an unlocked position;

FIG. 3C is a partial top plan view of the anterior cervical plate assembly of FIG. 1 in an unlocked position;

FIG. 4 is a top partial horizontal cross-section of the anterior cervical plate assembly of FIG. 1 taken along lines A-A;

FIG. 5 is a partial side perspective view of the anterior cervical plate assembly of FIG. 1 with a locking piece and fasteners omitted for purposes of illustration;

FIG. 6 is a partial bottom perspective view of the anterior cervical plate assembly of FIG. 1 in an unlocked position with fasteners omitted for purposes of illustration;

FIG. 7 is a perspective view of a blocking plate of the anterior cervical plate assembly of FIG. 1;

FIG. 8A is a bottom perspective view of a locking piece of the anterior cervical plate assembly of FIG. 1; and

FIG. 8B is a bottom plan view of the locking piece of FIG. 8A.

### DETAILED DESCRIPTION OF THE INVENTION

Reference will now be made in detail to the exemplary embodiments of the invention illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, above, below and diagonal, are used with respect to the accompanying drawings. Such directional terms used in conjunction with the following description of the drawings should not be construed to limit the scope of the invention in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges throughout this disclosure and various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

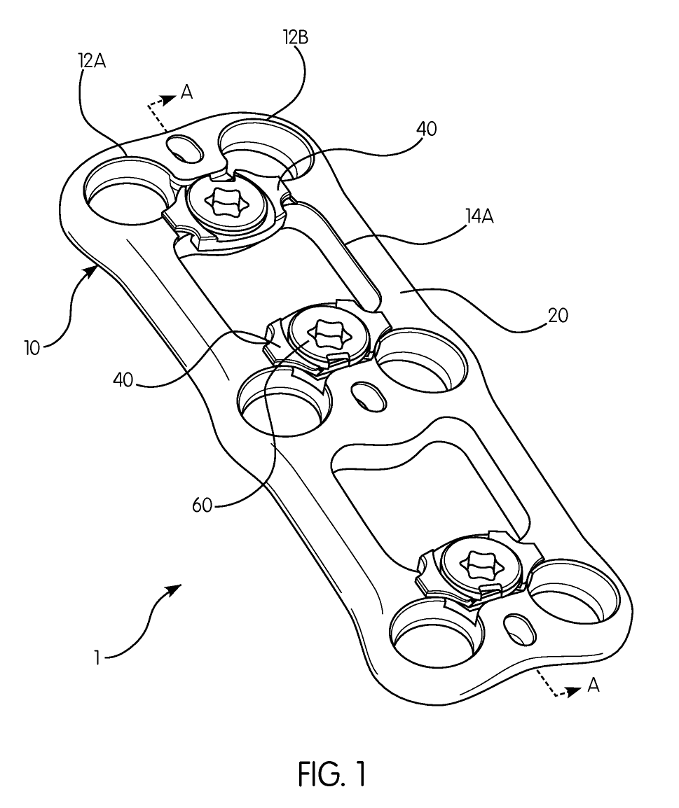

In accordance with a first exemplary embodiment, the present invention provides an anterior cervical plate assembly 1, as shown in FIG. 1. The anterior cervical plate assembly 1 includes a base plate 10, a blocking plate 40 or 40′, and a locking piece 70.

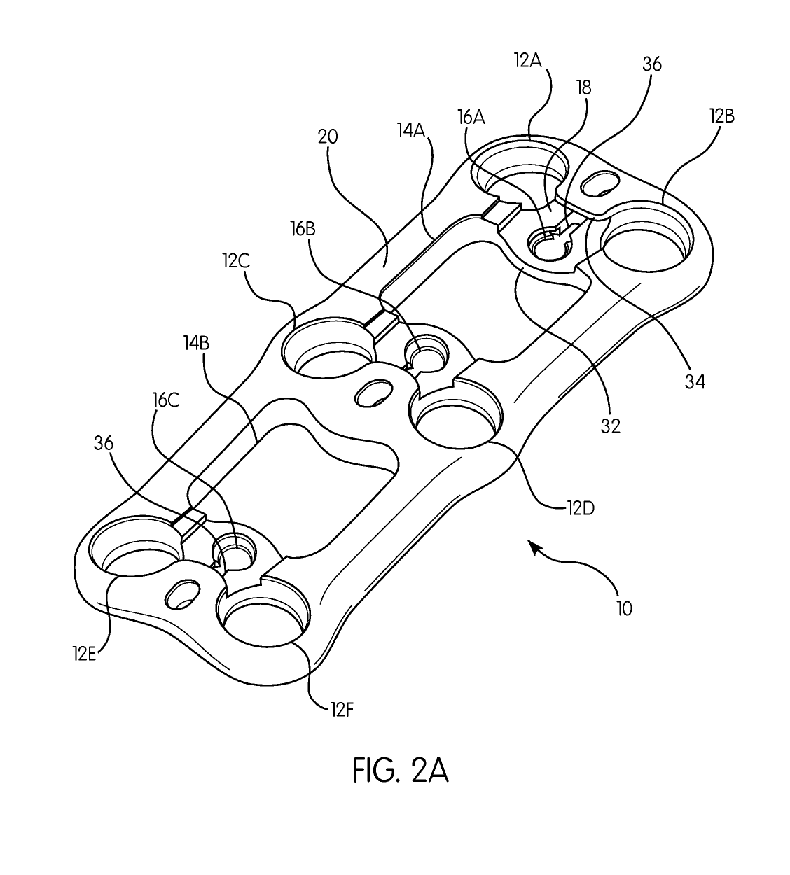

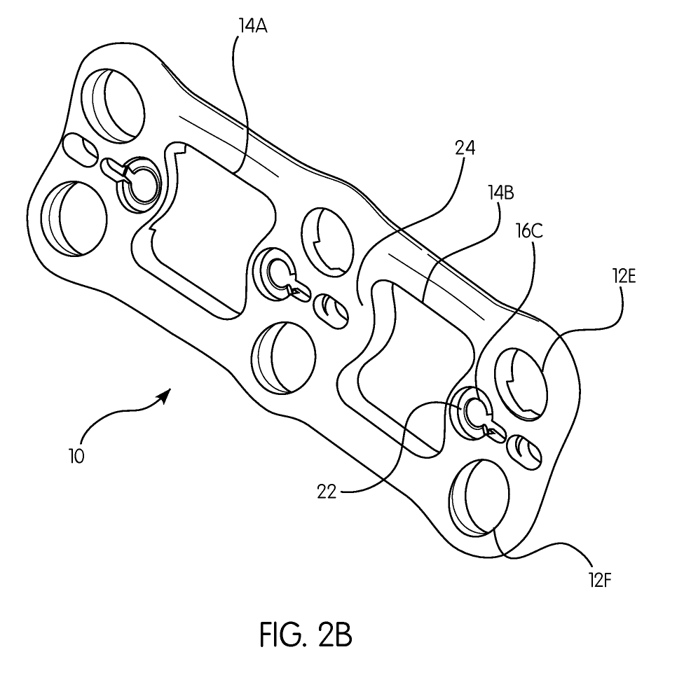

As best shown in FIG. 2A, the base plate 10 includes at least one opening 12, an elongated central aperture 14, and a through hole or fastening aperture 16. The base plate 10 also includes a top recess 18 formed about a top surface 20 that circumscribes through hole 16. As shown in FIG. 2B, base plate 10 further includes a bottom recess 22 from a bottom surface 24 that also circumscribes through hole 16.

Base plate 10 is generally rectangular, as shown in FIG. 1. The base plate 10 is illustrated as generally planar, although it may be curved laterally or longitudinally. For example, from the FIG. 1 perspective, left and right lateral edges of base plate 10 may be curved inwardly such that the plate is convex from a top perspective.

Openings 12A-F of base plate 10 are preferably circular as shown in FIG. 2A. The elongate apertures 14A-B are illustrated as generally rectangular with a central reduced waist portion and lateral flared ends. As shown in FIG. 2A, through holes 16A-C are generally circular. Other shapes are possible for the openings 12A-F, elongate apertures 14A-B and through holes 16A-C. Each of the openings 12A-F, elongate aperture 14A-B and through holes 16A-C extends completely through base plate 10.

In the first exemplary embodiment, base plate 10 includes six openings 12A-F, two elongate apertures 14A-B and three through holes 16A-C, although more or fewer of each are contemplated. For example, an alternative embodiment may have four openings, one elongate aperture and two through holes.

The openings 12A-F are laterally and longitudinally aligned relative to the base plate. The openings, e.g., 12A and 12B, are paired so as to be spaced apart from each other, aligned along a central longitudinal axis of the base plate 10 relative to other pairs of the openings 12C, 12D and 12E, 12F, and adjacent a respective through hole 16A.

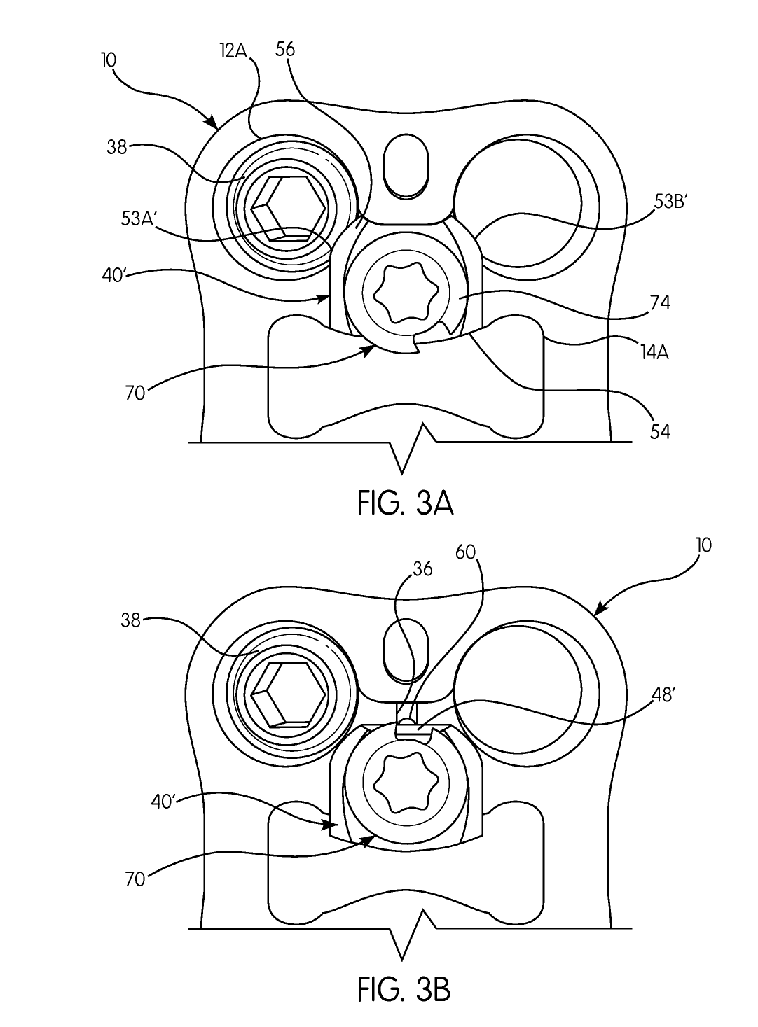

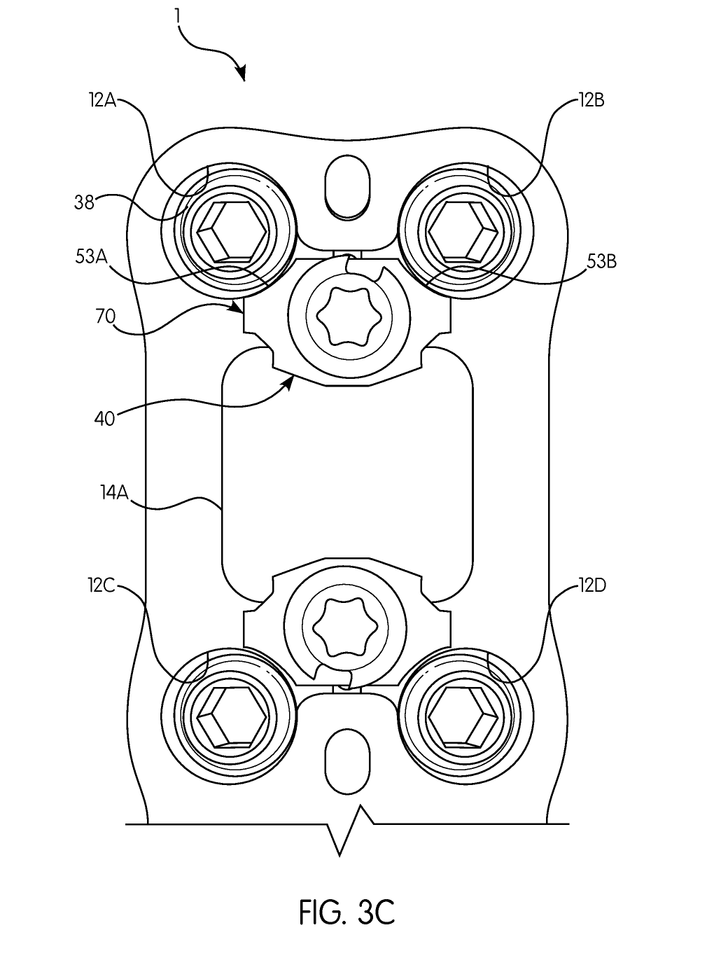

As shown in FIG. 3C, the elongate aperture 14A may be formed with opposite sides each adjacent to a pair of openings 12A, 12B and 12C, 12D, respectively.

Referring back to FIG. 2A, the arrangement and function of openings 12A, 12B with through hole 16A and elongate aperture 14A are substantially similar to the arrangement and function of other openings, through holes and elongate apertures in the anterior cervical plate assembly. As such, only the structure around openings 12A, 12B and elongate aperture 14A will be discussed in detail below.

In accordance with an exemplary embodiment, the through holes 16 are preferably spaced apart and aligned along a central longitudinal axis of the base plate 10. The through hole 16A is positioned generally between a pair of openings 12A, 12B and it is longitudinally offset from the pair of openings. Additionally, through hole 16A is positioned adjacent to aperture 14A and to each of the pair of openings 12A, 12B.

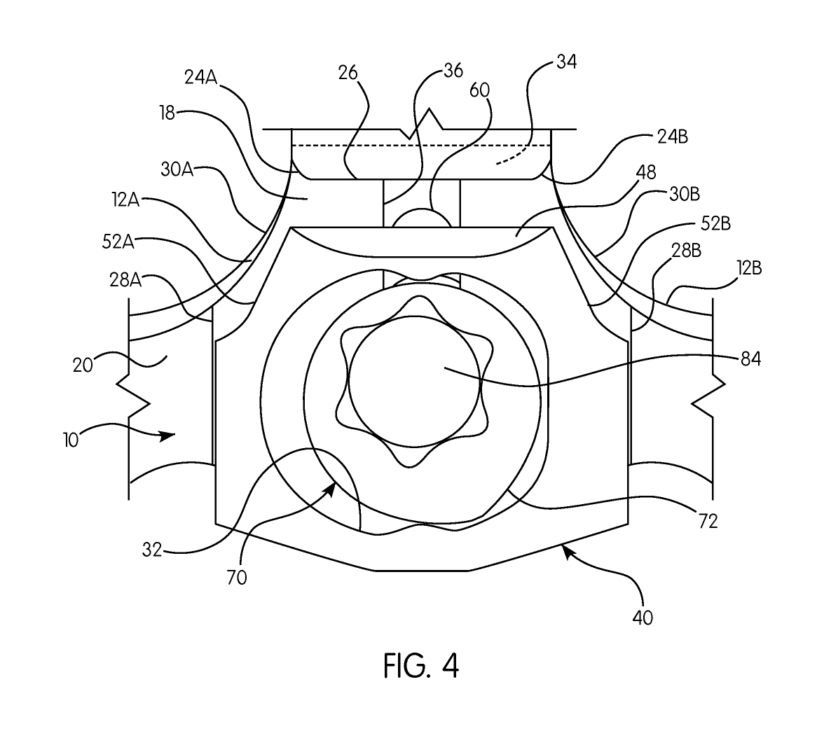

Referring to FIG. 4, the top recess 18 is inset from the top surface 20 of the base plate 10 and positioned between a pair of openings 12A-B. Above the top recess 18, top surface 20 of the base plate 10 includes upper corners 24A and 24B at either side of an upper lip 26. Top recess 18 includes sides 28A and 28B, edges 30A and 30B, and lower edge 32. The top recess 18 is generally sized and shaped to slidably receive the blocking plate 40 or 40′, as further described below.

The edges 30A, 30B of the top recess 18 each form part of a perimeter of one of the openings 12A, 12B of the base plate 10. Similarly, the lower edge 32 of the top recess 18 forms part of a perimeter of elongate aperture 14A. As such, the top recess 18 may border two openings 12A, 12B and border elongated aperture 14A. This arrangement of the top recess 18 allows for the blocking plate to selectively interact with the two openings 12A, 12B and elongated aperture 14A, as further described below.

Referring to FIG. 2B, the bottom recess 22 of the base plate 10 is inset from the bottom surface 24. Bottom recess 22 is concentric with the through hole 16 and generally configured as a counter-bore. The recess has a generally planar base at a constant depth from the bottom surface 24.

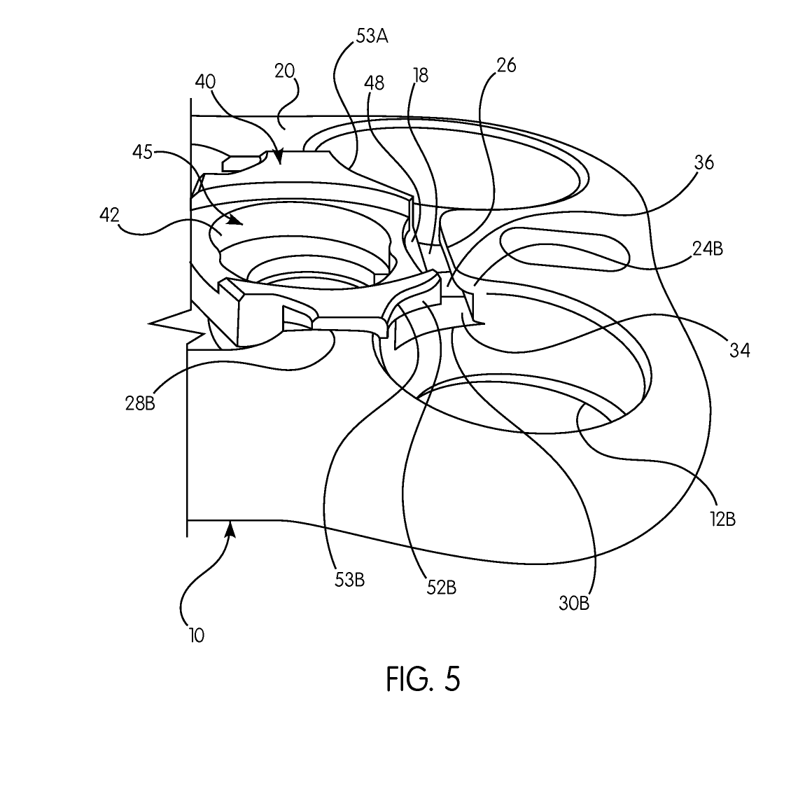

As shown in FIG. 5, the base plate 10 is configured to have an undercut 34 formed by the upper lip 26 of the base plate. The undercut 34 may have generally vertical and horizontal walls so as to form a generally rectangular void. An alternative undercut 34′ may be configured as generally wedge shaped such that the height of the undercut decreases as the undercut extends away from top recess 18, to correspond with the shape of relief cut 48′ shown in FIG. 3B.

As shown in FIGS. 2A and 4, the through hole 16A of the base plate 10 includes a guide or keyhole slot 36 extending radially from the through hole 16 toward the undercut 34 of the base plate. The guide slot 36 is elongated and receives therein a guide 60 of the blocking plate 40, as further described below.

As shown in FIG. 3C, a fastener 38 is seated in opening 12A of the base plate 10. The fastener 38 is preferably a bone screw or other anchor device suitable for seating in the vertebrae of a patient. The top perimeter of the fastener is preferably rounded or beveled. The anterior cervical plate assembly 1 may be provided with one fastener for each opening 12A-F; for example, six fasteners 38 may be provided for the exemplary base plate 10.

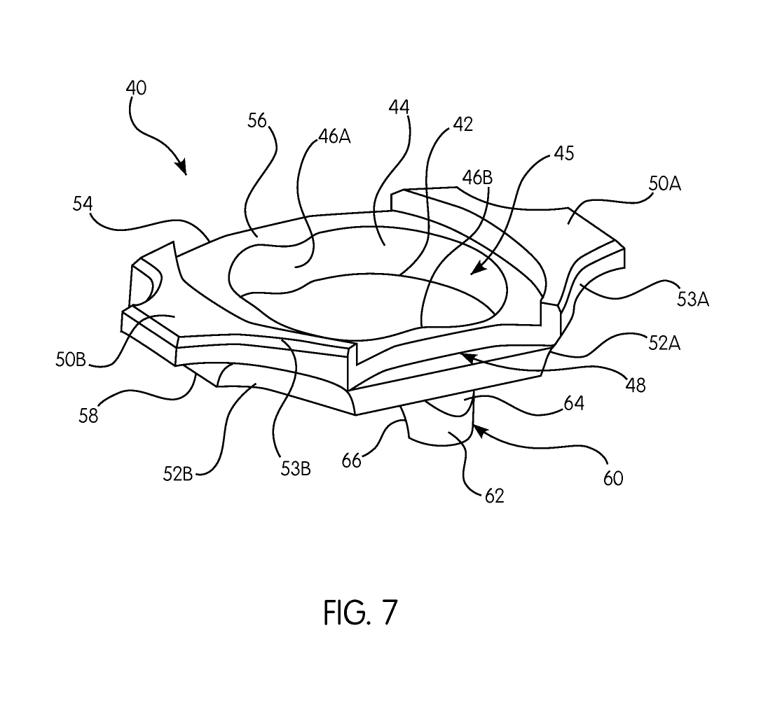

The blocking plate 40 is configured as best shown in FIG. 7 and includes a through hole or blocking plate aperture 42 with an inner face 44 having protrusions 46A, 46B. Blocking plate 40 further includes a relief portion 48, sidewalls 50A, 50B, one or more undercuts 52A, 52B forming overhangs 53A, 53B, a trailing edge 54, a top surface 56, a bottom surface 58, and the guide 60. An alternative embodiment of the blocking plate 40′ includes different shapes for relief portion 48′ and overhangs 53A′, 53B′, as shown in FIGS. 3A and 3B.

Referring to FIG. 7, the through hole 42 of the blocking plate 40 has an irregular but generally oval shape such that the inner face 44 in combination with the protrusions 46A, 46B define a cam follower 45. Each protrusion 46A, 46B extends radially inwardly toward a center of the through hole 42 and the protrusions are located substantially diametrically opposed to each other.

The relief portion 48 is cut below the top surface 56 of the blocking plate 40. The relief portion 48 may have various shapes suitable for receiving and engaging with the undercut 34 of the base plate. The relief portion 48 may be formed by substantially vertical walls so as to form a detent or protrusion. From a top perspective, the relief portion 48 may be slightly U-shaped between overhangs 53A, 53B. In an alternative embodiment, as best shown in FIG. 3B, the relief portion 48′ may be a relief cut sloped from an inner area to a distal end of the blocking plate 40′. This sloped relief portion 48′ may be a straight, concave or convex, preferably in correspondence with the shape of undercut 34′ of the base plate 10.

Referring back to FIG. 7, the sidewalls 50A, 50B extend along a length of the blocking plate 40 about its lateral sides and curve inwardly toward relief portion 48. The sidewalls 50A, 50B may be convex to curve inwardly, or they may be concave as shown in the blocking plate 40′ embodiment of FIG. 3A. The sidewalls 50A, 50B may have a substantially flat outermost side and taper toward the trailing edge 54, as shown in FIG.

7.

The undercuts 52A, 52B are formed out of the corners of the blocking plate 40 below the relief portion 48 and below overhangs 53A, 53B (or 53A′, 53B′ for blocking plate 40′). The undercuts 52A, 52B are three-dimensionally convex, i.e., generally hemispherical. As shown in FIGS. 4 and 5, a lower portion of the undercuts 52A, 52B may generally or substantially match the shape of corresponding edges 30A, 30B of the base plate 10 so as to mimic the shape of the perimeter of the openings 12A, 12B. Likewise, the undercuts 52A, 52B are shaped to correspond to the shape of a fastener 38 to enable a close fit against the fastener. Extending upwardly in the FIG. 5 perspective, the undercuts 52A, 52B also extend distally away from the blocking plate plate 40.

Overhangs 53A, 53B extend radially outwardly from the blocking plate 40 so as to cover and extend from the undercuts 52A, 52B. As further discussed below, the overhangs 53A, 53B are sized and shaped to partially overlap and obstruct the openings 12A, 12B, respectively. Consequently, the overhangs 53A, 53B overlap and obstruct fasteners 38 placed in openings 12A and 12B. The overhangs 53A, 53B are generally concave as shown in FIG. 7, while overhangs 53A′, 53B′ are generally convex as shown in FIG. 3A.

The trailing edge 54 of the blocking plate 40 is preferably curved convexly, as best shown in FIG. 3C, with a curvature similar to that of the elongate aperture 14A. The top surface 56 and bottom surface 58 of the blocking plate may be generally flat, generally planar and generally parallel to each other, as shown in FIG. 5.

Referring back to FIG. 7, the guide 60 of the blocking plate 40 extends below the bottom surface 58 and forward beyond the relief portion 48. A front face 62 of the guide 60 can be generally vertical and generally convex so as to form an overall half cylindrical shape. A top portion 64 may be angled so as to form a transitional slope from the front face 62 rearwardly to the rest of the blocking plate 40. A rear surface 66 of the guide 60 may be convexly curved.

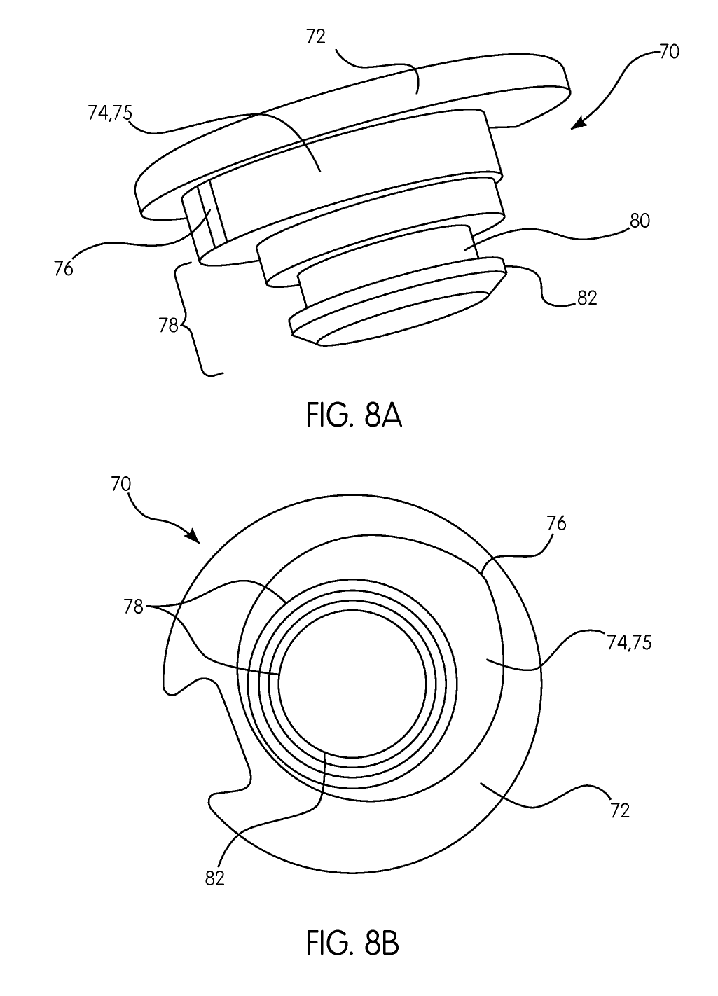

The locking piece 70 is configured as best shown in FIG. 8A and includes a top portion 72 above a reduced diameter portion 74. The reduced diameter portion 74 defines a cam 75 having a substantially flat end 76, also referred to as a cam surface or a crest. The cam 75 extends along a substantially circumferential direction of the locking piece 70 with the flat end 76. The locking piece 70 further includes a stem portion or lower portion 78 below the reduced diameter portion 74, the stem portion having a reduced waist portion or annular groove 80 and a foot portion 82. The locking piece 70 is configured to extend through both the through hole 42 of the blocking plate 40 and the through hole 16 of the base plate 10.

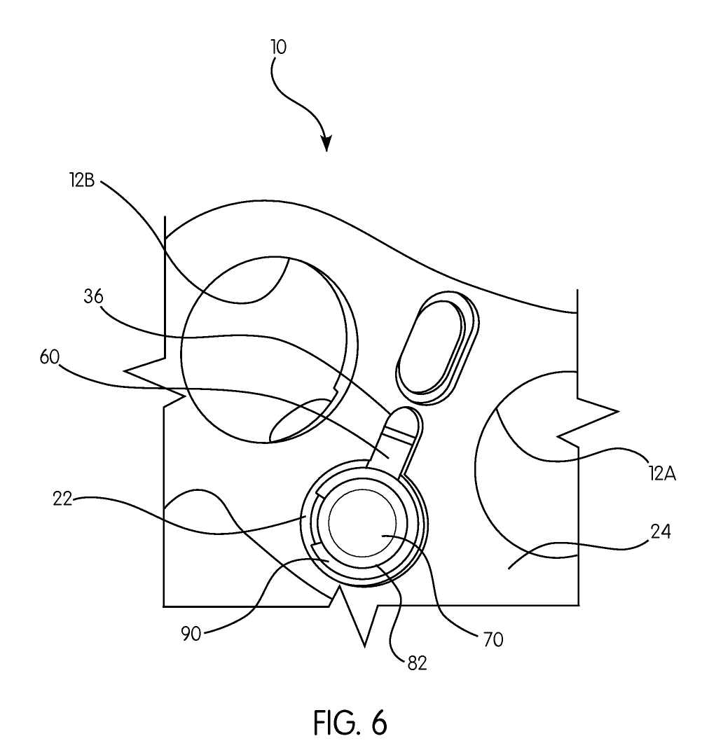

As shown in FIG. 6, a retaining member 90 is provided. The retaining member 90 is preferably ring shaped, in particular C-shaped, O-shaped, or split. In other words, the retaining member 90 may be a locking ring, C-ring, O-ring or a split ring. The retaining member 90 is illustrated as generally planar and thin. Other ring shapes or equivalent devices may also provide the functionality of retaining member 90.

In operation, the base plate 10, blocking plate 40, locking piece 70 and retaining member 90 are complementarily sized and shaped. The shapes discussed herein are exemplary; other shapes are possible so long as they are generally complementary such that the anterior cervical plate assembly 1 functions similarly.

When assembled, various connecting parts of the anterior cervical plate assembly are sized so as to be generally flush and minimize the overall depth of the assembly. For example, as shown in FIGS. 1, 3A and 3B, the depth of the top surface 72 of the locking piece 70 is less than or equal to a depth in the blocking plate 40 defined between sidewalls 50A, 50B and top surface 56. Additionally, the depth of the retaining member 90 is preferably less than or equal to the depth of the reduced waist portion 80 of the stem portion 78 of the locking piece 70.

Referring to FIGS. 5 and 7, the top recess 18 of the base plate 10 is generally flat and planar to correspond to the bottom surface 58 of the blocking plate 40. Similarly, the top surface 56 of the blocking plate is generally flat and planar to correspond with an underside of the top portion 72 of the locking piece 70, as shown in FIGS. 7 and 8.

The retaining member 90 is sized with an inner diameter that is less than the diameter of the stem portion 78 and foot portion 82 of the locking piece 70 but slightly smaller than, equal to or greater than the diameter of reduced waist portion 80. Further, the retaining member 90 outer diameter is greater than the diameter of through hole 16 of base plate 10. With these relative dimensions, the retaining member 90 functions to hold the locking piece 70 within through hole 16 by encircling the reduced waist portion 80. Thus the retaining member 90 functions to prevent back-out or longitudinal movement of the locking piece. By the same arrangement, the retaining member 90 secures the locking piece 70 to the blocking plate 40 or 40′. At the same time, the retaining member 90 allows the locking piece 70 to rotate.

The blocking plate 40 or 40′ is configured to adopt a locked position in which the blocking plate overlaps the opening 12A, shown in FIG. 3A. The blocking plate 40 or 40′ may adopt this locked position when the fastener 38 is positioned within the opening. In particular, when the blocking plate 40 or 40′ is in the locked position, the sidewalls 50A, 50B and undercuts 52A, 52B of the blocking plate may overlap the openings 12A, 12B and the fasteners 38. As such, undercuts 52A, 52B are positioned closely adjacent the fasteners 38 due to the convex shape of the undercuts while the overhangs 53A, 53B extend to a position over openings 12A, 12B respectfully to prevent fasteners 38 in the openings from backing out of the openings. In the exemplary embodiment of FIG. 3B, the blocking plate 40′ overlaps two openings 12A, 12B (and two fasteners 38) when in the locked position.

As shown in FIG. 5, the relief portion 48 of the blocking plate 40 and the undercut 34 of the base plate 10 are shaped to fit together as a complementary detent and recess. Alternatively with blocking plate 40′, the relief portion 48′ and undercut 34′ are shaped as complementarily angled slopes, as shown in FIG. 3B.

Likewise the blocking plate 40 or 40′ is configured to adopt an unlocked position in which the opening 12 is unobstructed such that a fastener 38 may be freely inserted or removed, as shown in FIGS. 3B and 3C. In this unlocked position, the relief portion 48 is spaced from upper lip 26 and overhangs 53A, 53B are not positioned over openings 12A, 12B.

As best shown in FIG. 4, the guide 60 of the blocking plate 40 or 40′ slides within the guide slot 36 of the base plate 10 when the blocking plate moves between its locked and unlocked positions. When the blocking plate 40 or 40′ moves from the unlocked to the locked position, the slope of the front face 62 and top surface 64 of the guide 60 aid in aligning the blocking plate 40 or 40′ with the base plate 10. Proper alignment of the guide 60 within the guide slot 36 ensures that the relief portion 48 or 48′ of the base plate 40 or 40′ engages the undercut 34 or 34′ of the base plate 10 to attain the locked position.

The locking piece 70 functions to actuate the movement of the blocking plate 40 or 40′ between the locked and unlocked positions. Rotation of the locking piece 70 causes the cam 75 to cam against the cam follower 45 of the blocking plate 40 causing translational movement of the blocking plate 40 or 40′ along a longitudinal direction of the baseplate. The rotation of the locking piece 70 can be effected by a screwdriver, Allen wrench, or the like. The locking piece 70 rotates approximately 180 degrees between the locked and unlocked positions. The locking piece 70 is rotated in opposite directions (clockwise and counter-clockwise) to move between the locked and unlocked positions.

During longitudinal movement of the blocking plate 40 or 40′ as caused by rotation of the locking piece 70, as best shown in FIG. 5, the sidewalls 50A, 50B of the blocking plate slide along sides 28A, 28B of the base plate 10, the guide 60 of the blocking plate slides within the guide slot 36 of the base plate and the relief portion 48 or 48′ slides within undercut 34 or 34′.

The locking piece 70 functions to retain the blocking plate 40 in either the locked or unlocked positions. Once a fully locked or fully unlocked position has been achieved, the flat end 76 of the locking piece 70 aligns and frictionally engages or otherwise mechanically locks with one of the protrusions 46A, 46B of the blocking plate such that incidental forces on the blocking plate do not result in translation of the blocking plate. In the exemplary embodiment, the locking piece 70 cannot rotate past either protrusion 46A, 46B, such that it is impossible for the locking piece to rotate a full 360 degrees. Other embodiments are contemplated allowing more or less rotation, so long as the locking piece 70 can mechanically lock in either the locked or unlocked position.

The trailing edge 54 of the blocking plate 40 or 40′ is sized and shaped so as to not obstruct the elongate opening 14A when the blocking plate is in the locked position, as shown in FIG. 3A.

The anterior cervical plate assembly functions to retain the fastener 38 in place and prevent backing out of the fastener after being implanted in the patient. The blocking plate 40 or 40′ is held in place vertically via the undercut 34 or 34′ of the base plate 10, which engages the relief portion 48 or 48′ of the blocking plate. At the same time, the blocking plate 40 is held in place longitudinally along the base plate due to its protrusions 46A, 46B engaging the flat end 76 of the locking piece 70.

It will be appreciated by those skilled in the art that changes could be made to the embodiments described above without departing from the broad inventive concept thereof. For example, the anterior cervical plate may be designed to receive from one to five fasteners 38, instead of six as illustrated. In such a case, a blocking plate 40 may be designed with only one undercut 52A or 52B to engage a single fastener 38. The base plate 10, blocking plate 40, and locking piece 70 may be formed from multiple parts instead of the integrally formed constructions illustrated for each. As another example, the respective dimensions and shapes of the through hole 42, the protrusions 46A, 46B, the cam-shaped reduced diameter portion 74, and flat end 76 may be varied so long as they cause movement of the blocking plate to the locked position and/or hold the blocking plate in at least one of the unlocked and locked positions. It is to be understood, therefore, that this invention is not limited to the particular embodiment disclosed, but it is intended to cover modifications within the spirit and scope of the present invention as defined by the appended claims.

I claim:

1. An anterior cervical plate assembly comprising:
a base plate including a recess;
a blocking plate positioned within the recess, the blocking plate including:
an aperture, and
a protrusion extending from a surface of the blocking plate forming the aperture; and
a locking piece positioned within the aperture, the locking piece including a cam for engaging the protrusion.

2. The anterior cervical plate assembly of claim 1, wherein the locking piece is configured to engage the blocking plate to move the blocking plate between first and second positions.

3. The anterior cervical plate assembly of claim 2, wherein the cam of the locking piece is configured to engage the protrusion of the blocking plate to move the blocking plate between the first and second positions.

4. The anterior cervical plate assembly of claim 1, wherein the cam includes a flat end for frictionally engaging the protrusion of the blocking plate.

5. The anterior cervical plate assembly of claim 1, wherein the cam extends along a substantially circumferential direction of the locking piece.

6. The anterior cervical plate assembly of claim 1, wherein the locking piece further includes:
a top portion;
a reduced diameter portion adjacent the top portion, wherein the reduced diameter portion includes the cam; and
a stem portion extending from the reduced diameter portion.

7. The anterior cervical plate assembly of claim 6, wherein the stem includes a reduced waist portion for receiving a retaining member for securing the locking piece to the blocking plate

8. The anterior cervical plate assembly of claim 1, further comprising a retaining member for engaging the locking piece and securing the locking piece to the blocking plate.

9. The anterior cervical plate assembly of claim 8, wherein the retaining member is an O-ring, a split ring or a C-ring.

10. The anterior cervical plate assembly of claim 1, wherein the blocking plate includes at least two protrusions.

11. The anterior cervical plate assembly of claim 10, wherein the at least two protrusions are positioned substantially diametrically opposed to each other.

12. The anterior cervical plate assembly of claim 2, wherein the base plate further includes a pair of openings for receiving a respective fixation device, the recess is positioned between the pair of openings, and the blocking plate prevents the fixation device from disengaging from the base plate when in the second position.

13. The anterior cervical plate assembly of claim 2, wherein the base plate further includes a pair of openings for receiving a respective fixation device, and the blocking plate overlaps the pair of openings in the second position.

14. The anterior cervical plate assembly of claim 1, wherein the blocking plate further including a relief flange portion and the base plate further includes an undercut adjacent to the recess for receiving the relief flange portion.

15. The anterior cervical plate assembly of claim 1, wherein the base plate further includes an angled undercut adjacent to the recess, and the blocking plate further includes a complementarily angled relief cut configured to engage under the undercut.

16. The anterior cervical plate assembly of claim 2, wherein the base plate further includes:
a pair of openings for receiving a respective fixation device; and
an elongated aperture adjacent the pair of openings.

17. The anterior cervical plate assembly of claim 16, wherein the blocking plate extends over the elongated aperture when in the first position.

18. An anterior cervical plate assembly comprising:
a base plate that includes:
a pair of openings, and
a recess between the pair of openings;
a blocking plate positioned within the recess, the blocking plate including:
a through hole defining a cam follower; and
a locking piece positioned within the aperture of the blocking plate, the locking piece including:
a top portion;
a reduced diameter portion adjacent the top portion, wherein the reduced diameter portion includes a cam, and
a stem portion extending from the reduced diameter portion.

19. The anterior cervical plate assembly of claim 18, wherein the cam follower includes a pair of protrusions extending from a wall defining the through hole

20. An anterior cervical plate assembly, comprising:
a base plate including:
a fastening aperture having a top recess, a bottom recess and an inner diameter,
first and second apertures for receiving bone screws,
an elongated central aperture,
a top surface and a bottom surface, and
an undercut extending below the top surface and between the first and second apertures;
a blocking plate positioned in the top recess, configured to adopt an unlocked position wherein the first and second apertures are unobstructed and configured to adopt a locked position obstructing the first and second apertures, the blocking plate including:
a blocking plate aperture,
first and second protrusions extending into the blocking plate aperture,
a relief cutout configured to slide under the undercut in the locked position, and
a pair of outer corners each configured to obstruct one of the first and second base plate apertures in the locked position;
a locking piece extending through both the fastening aperture and the blocking plate aperture, including:
a top portion positioned upon the blocking plate,
a cam-shaped portion configured for engaging the protrusions, the cam-shaped portion having a flat end, and
a lower portion having a reduced waist; and
a locking ring positioned within the bottom recess, the locking ring being sized to encircle the reduced waist of the locking piece so as to prevent removal of the locking piece from the fastening aperture.
